SEQUENCE LISTING (25 sequences)

SEQ ID NO: 1 - protein, 27 aa, Homo sapiens
HSDGIFTDSYSRYRKQMAVKKYLAAVL

SEQ ID NO: 2 - protein, 28 aa, Homo sapiens
HSDAVFTDNYTRLRKQMAVKKYLNSILN

SEQ ID NO: 3 - protein, 27 aa, Artificial Sequence
  PACAP glycopeptide analog
  MISC_FEATURE (2)..(2): D-Ser or L-Ser
  MISC_FEATURE (9)..(9): Ser may be glycosylated
  MISC_FEATURE (11)..(11): Ser may be glycosylated
  MISC_FEATURE (17)..(17): M or Nle
  MISC_FEATURE (27)..(27): L or S
  MISC_FEATURE (27)..(27): Xaa may be glycosylated Ser
HSDGIFTDSYSRYRKQXAVKKYLAAVX

SEQ ID NO: 4 - protein, 28 aa, Artificial Sequence
  VIP glycopeptide analog
  MISC_FEATURE (2)..(2): D-Ser or L-Ser
  MISC_FEATURE (17)..(17): M or Nle
  MISC_FEATURE (25)..(25): Ser may be glycosylated
HSDAVFTDNYTRLRKQXAVKKYLNSILN

SEQ ID NO: 5 - protein, 27 aa, Artificial Sequence
  PACAP glycopeptide analog
  MISC_FEATURE (2)..(2): D-Ser or L-Ser
  MISC_FEATURE (9)..(9): Glycosylated Ser
HSDGIFTDSYSRYRKQMAVKKYLAAVL

SEQ ID NO: 6 - protein, 27 aa, Artificial Sequence
  PACAP glycopeptide analog
  MISC_FEATURE (2)..(2): D-Ser or L-Ser
  MISC_FEATURE (11)..(11): Glycosylated Ser
HSDGIFTDSYSRYRKQMAVKKYLAAVL

SEQ ID NO: 7 - protein, 27 aa, Artificial Sequence
  PACAP glycopeptide analog
  MISC_FEATURE (2)..(2): D-Ser or L-Ser
  MISC_FEATURE (17)..(17): Nle
HSDGIFTDSYSRYRKQXAVKKYLAAVL

SEQ ID NO: 8 - protein, 27 aa, Artificial Sequence
  PACAP glycopeptide analog
  MISC_FEATURE (2)..(2): D-Ser or L-Ser
  MISC_FEATURE (9)..(9): Glycosylated Ser
  MISC_FEATURE (17)..(17): Nle
HSDGIFTDSYSRYRKQXAVKKYLAAVL

SEQ ID NO: 9 - protein, 27 aa, Artificial Sequence
  PACAP glycopeptide analog
  MISC_FEATURE (2)..(2): D-Ser or L-Ser
  MISC_FEATURE (11)..(11): Glycosylated Ser
  MISC_FEATURE (17)..(17): Nle
HSDGIFTDSYSRYRKQXAVKKYLAAVL

SEQ ID NO: 10 - protein, 27 aa, Artificial Sequence
  PACAP glycopeptide analog
  MISC_FEATURE (2)..(2): D-Ser or L-Ser
  MISC_FEATURE (17)..(17): Nle
HSDGIFTDSYSRYRKQXAVKKYLAAVS

SEQ ID NO: 11 - protein, 27 aa, Artificial Sequence
  PACAP glycopeptide analog
  MISC_FEATURE (2)..(2): D-Ser or L-Ser
  MISC_FEATURE (9)..(9): Glycosylated Ser
  MISC_FEATURE (17)..(17): Nle
HSDGIFTDSYSRYRKQXAVKKYLAAVS

SEQ ID NO: 12 - protein, 27 aa, Artificial Sequence
  PACAP glycopeptide analog
  MISC_FEATURE (2)..(2): D-Ser or L-Ser
  MISC_FEATURE (11)..(11): Glycosylated Ser
  MISC_FEATURE (17)..(17): Nle
HSDGIFTDSYSRYRKQXAVKKYLAAVS

SEQ ID NO: 13 - protein, 27 aa, Artificial Sequence
  PACAP glycopeptide analog
  MISC_FEATURE (2)..(2): D-Ser or L-Ser
  MISC_FEATURE (17)..(17): Nle
  MISC_FEATURE (27)..(27): Glycosylated Ser
HSDGIFTDSYSRYRKQXAVKKYLAAVS

SEQ ID NO: 14 - protein, 28 aa, Artificial Sequence
  VIP glycopeptide analog
  MISC_FEATURE (2)..(2): D-Ser or L-Ser
  MISC_FEATURE (25)..(25): Glycosylated Ser
HSDAVFTDNYTRLRKQMAVKKYLNSILN

SEQ ID NO: 15 - protein, 28 aa, Artificial Sequence
  VIP glycopeptide analog
  MISC_FEATURE (2)..(2): D-Ser or L-Ser
  MISC_FEATURE (17)..(17): Nle
HSDAVFTDNYTRLRKQXAVKKYLNSILN

SEQ ID NO: 16 - protein, 28 aa, Artificial Sequence
  VIP glycopeptide analog
  MISC_FEATURE (2)..(2): D-Ser or L-Ser
  MISC_FEATURE (17)..(17): Nle
  MISC_FEATURE (25)..(25): Glycosylated Ser
HSDAVFTDNYTRLRKQXAVKKYLNSILN

SEQ ID NO: 17 - protein, 28 aa, Artificial Sequence
  PACAP glycopeptide analog
  MISC_FEATURE (28)..(28): Ser(Lactose)
HSDGIFTDSYSRYRKQLAVKKYLAAVLS

SEQ ID NO: 18 - protein, 28 aa, Artificial Sequence
  PACAP glycopeptide analog
  MISC_FEATURE (28)..(28): Ser(Melibiose)
HSDGIFTDSYSRYRKQLAVKKYLAAVLS

SEQ ID NO: 19 - protein, 28 aa, Artificial Sequence
  PACAP glycopeptide analog
  MISC_FEATURE (28)..(28): Ser(Cellobiose)
HSDGIFTDSYSRYRKQLAVKKYLAAVLS

SEQ ID NO: 20 - protein, 28 aa, Artificial Sequence
  PACAP glycopeptide analog
  MISC_FEATURE (2)..(2): D-Ser
  MISC_FEATURE (17)..(17): Norvaline
  MISC_FEATURE (28)..(28): L-Serine-beta-D-Glucoside
HSDGIFTDSYSRYRKQXAVKKYLAAVLS

SEQ ID NO: 21 - protein, 27 aa, Artificial Sequence
  PACAP glycopeptide analog
  MISC_FEATURE (2)..(2): D-Ser
  MISC_FEATURE (17)..(17): Norvaline
  MISC_FEATURE (27)..(27): L-Serine-beta-D-Glucoside
HSDGIFTDSYSRYRKQXAVKKYLAAVS

SEQ ID NO: 22 - protein, 27 aa, Artificial Sequence
  PACAP glycopeptide analog
  MISC_FEATURE (2)..(2): D-Ser
  MISC_FEATURE (17)..(17): Norvaline
  MISC_FEATURE (26)..(26): L-Serine-beta-D-Glucoside
HSDGIFTDSYSRYRKQXAVKKYLAASL

SEQ ID NO: 23 - protein, 28 aa, Artificial Sequence
  PACAP glycopeptide analog
  MISC_FEATURE (2)..(2): D-Ser
  MISC_FEATURE (17)..(17): Norvaline
  MISC_FEATURE (28)..(28): L-Serine-beta-D-Lactoside
HSDGIFTDSYSRYRKQXAVKKYLAAVLS

SEQ ID NO: 24 - protein, 27 aa, Artificial Sequence
  PACAP glycopeptide analog
  MISC_FEATURE (2)..(2): D-Ser
  MISC_FEATURE (17)..(17): Norvaline
  MISC_FEATURE (27)..(27): L-Serine-beta-D-Lactoside
HSDGIFTDSYSRYRKQXAVKKYLAAVS

SEQ ID NO: 25 - protein, 27 aa, Artificial Sequence
  PACAP glycopeptide analog
  MISC_FEATURE (2)..(2): D-Ser
  MISC_FEATURE (17)..(17): Norvaline
  MISC_FEATURE (26)..(26): L-Serine-beta-D-Lactoside
HSDGIFTDSYSRYRKQXAVKKYLAASL

(19) United States
(12) Patent Application Publication
(10) Pub. No.: US 2022/0380431 A1
(43) Pub. Date: Dec. 1, 2022

(54) GLYCOPEPTIDE ANALOGS OF SECRETIN FAMILY PEPTIDES

(71) Applicant: ARIZONA BOARD OF REGENTS ON BEHALF OF THE UNIVERSITY OF ARIZONA, Tucson, AZ (US)

(72) Inventors: Robin L. Polt, Tucson, AZ (US); Christopher Apostol, Tucson, AZ (US); Michael L. Heien, Tucson, AZ (US); Chenxi Liu, Tucson, AZ (US); John M. Streicher, Tucson, AZ (US); Lajos Z. Szabo, Tucson, AZ (US); Torsten Falk, Tucson, AZ (US)

(21) Appl. No.: 16/637,702

(22) PCT Filed: Aug. 9, 2018
(86) PCT No.: PCT/US18/46136

§ 371 Date: Feb. 7, 2020

Related U.S. Application Data
(60) Provisional application No. 62/543,152, filed on Aug. 9, 2017.

(51) Int. Cl. C07K 14/575; C07K 14/72

(52) U.S. Cl. CPC C07K 14/57563; C07K 14/72; C07K 2317/41

(57) ABSTRACT

Glycopeptide analogs of secretin family peptides, including PACAP and VIP, are described herein. These glycopeptides analogs can have neuroprotective properties and enhanced ability to cross the blood brain barrier (BBB) and/or enhanced stability. These glycosylated peptides can be used as drugs for treatment of CNS disorders, such as Parkinson's disease.

### CROSS REFERENCE

This application is a 371 and claims benefit of PCT/US18/46136 filed Aug. 9, 2018, which claims benefit of U.S. Provisional Application No. 62/543,152, filed Aug. 9, 2017, the specification(s) of which is/are incorporated herein in their entirety by reference.

### GOVERNMENT SUPPORT

This invention was made with government support under Grant No. R01 NS052727 awarded by National Institutes of Health, and Grant No. CHE0607917 awarded by NSF. The government has certain rights in the invention.

### REFERENCE TO SEQUENCE LISTING

Applicant asserts that the information recorded in the form of an Annex C/ST.25 text file submitted under Rule 13ter.1(a), entitled UNIA_17_35_PCT_Sequence_Listing_ST25, is identical to that forming part of the international application as filed. The content of the sequence listing is incorporated herein by reference in its entirety.

### FIELD OF THE INVENTION

The present invention relates to glycosylated analogs of secretin family peptides, including PACAP and VIP, for use as neuroprotective agents.

### BACKGROUND OF THE INVENTION

G-protein coupled receptors (GPCRs) are the largest class of transmembrane proteins which are involved in mediating a myriad of biological processes, making them popular drug targets. In fact, around 40% of drugs currently on the market target a GPCR. Pituitary adenylate cyclase-activating polypeptide type I receptor (PAC₁) and vasoactive intestinal peptide receptors, VPAC₁ and VPAC₂, are receptors of the secretin family of GPCRs. These three receptors are pleiotropic and widely distributed in the central nervous system (CNS) and periphery.

Pituitary adenylate cyclase activating peptide (PACAP1), which has the sequence: HSDGIFTDSY₁₀SRYRKQMAVK₂₀KYLAAVL (SEQ ID NO: 1), can bind to and activate the PAC₁, VPAC₁ and VPAC₂ receptors. Vasoactive intestinal peptide (VIP) is a 28 amino acid peptide with the sequence: HSDAVFTDNY₁₀TRLRKQMAVK₂₀KYLNSILN (SEQ ID NO: 2), which has 68% identity with PACAP₁₋₂₇, and activates the VPAC₁ and VPAC₂ receptors equally to the PACAP ligand, but is several orders of magnitude less active at the PAC₁ receptor. These peptide neuromodulators are potentially neurotrophic and/or neuroprotective peptides for treatment of CNS conditions, such as, for example, Parkinson's disease (PD), which is an idiopathic neurological disorder in which the dopaminergic neurons of the substantia nigra pars compacta (SNc) degenerate.

The blood-brain barrier (BBB) prevents entry into the brain of many drugs from the blood. Thus, the presence of the BBB can make the development of new treatments of brain diseases difficult. Glycosylation of peptide chains can improve penetration of the BBB to facilitate cerebral entry of the glycopeptides derivative and consequently, activation of the target receptor. It is possible that both PACAP and VIP interact strongly with biological membranes, and likely promotes the kinetics of binding once the glycosylated peptides arrive at the neuronal membrane by reducing the 3-dimensional search for the receptors to a 2-dimensional “membrane search”. This glycosylation approach may be promising for delivering PACAP and VIP drugs to the CNS for the treatment of PD.

The present invention features glycosylated peptide analogs of PACAP₁₋₂₇ or VIP₁₋₂₃ that have enhanced ability to cross the BBB and/or enhanced half-lives, and that can target specific GPCRs both in and outside of the CNS to treat several conditions, such as, for example, Parkinson's disease.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

It is an objective of the present invention to provide for glycopeptides that target the PAC₁, VPAC₁ and VPAC₂ receptors, along with methods of use thereof in treating CNS disorders, such as Parkinson's disease, as specified in the independent claims. One of the unique and inventive technical features of the present invention is the use of carbohydrates to modulate the amphipathicity of various peptides and ultimately enhance their BBB penetration, bioavailability, and enzymatic stability. Embodiments of the invention are given in the dependent claims. Embodiments of the present invention can be freely combined with each other if they are not mutually exclusive.

According to some aspects, the present invention features a glycopeptide analog of PACAP₁₋₂₇ (SEQ ID NO: 1) or VIP₁₋₂₃ (SEQ ID NO: 2). In some embodiments, the glycopeptide analog may comprise a sequence according to any one of the following:

(SEQ ID NO: 3)
HSDGIFTDSY₁₀SRYRKQX¹AVK₂₀KYLAAVX²;
or
(SEQ ID 4)
HSDAVFTDNY₁₀TRLRKQX¹AVK₂₀KYLNSILN.

In some embodiments, X¹ may be M or norleucine or norvaline. Without wishing to be bound by a theory, when X¹ is norleucine, the glycopeptide analog can have an increased stability as compared to the glycopeptide analog where X¹ is M. In other embodiments, X² may be L or S. In preferred embodiments, at least one of the S residues in the sequence may be glycosylated with a glycan.

According to other aspects, the glycopeptides analogs described herein may be utilized in pharmaceutical formulations and methods of treatment. In some embodiments, pharmaceutical compositions comprising the glycopeptides analogs may be effective for treating symptoms associated with Parkinson's disease. In other lycopepti, pharmaceutical compositions comprising the glycopeptides analogs may be effective for treating or preventing symptoms associated with degeneration of dopaminergic neurons of the substantia nigra pars compacta.

According to some other aspects, the present invention may feature a method of treating a symptom associated with Parkinson's disease in a subject. In other aspects, the present invention may feature a method of treating a symptom associated with degeneration of dopaminergic neurons of the substantia nigra pars compacta. Said methods may comprise administering to the subject a therapeutically effective amount of a composition comprising any of the glycopeptide analogs described herein, thereby alleviating the symptom.

### BRIEF DESCRIPTION OF THE DRAWINGS

The features and advantages of the present invention will become apparent from a consideration of the following detailed description presented in connection with the accompanying drawings in which:

FIG. 1A shows in vitro stability data for the PACAP analogs of the present invention in aqueous solution. The peptides are stable over hours. The sequences 2ls98la, 2ls98Mel, 2ls98cell, CRA3000, CRA3001, CRA3002, CRA3003, CRA3004, CRA3005 correspond to the lactoside, melibiose, and cellobioside modified versions of PACAP. DADLE is a non-endogenous peptide used as a control. The sequences in FIG. 1A are shown in TABLE 1 below.

FIG. 1B shows the stability in cerebral spinal fluid. The compounds break down at various rates corresponding to their modifications.

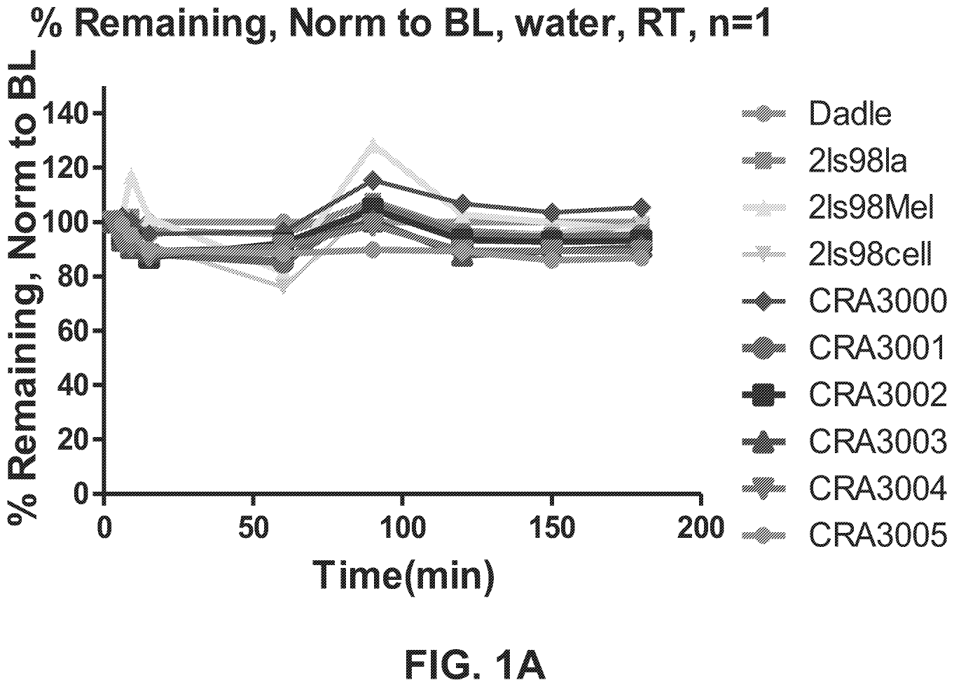

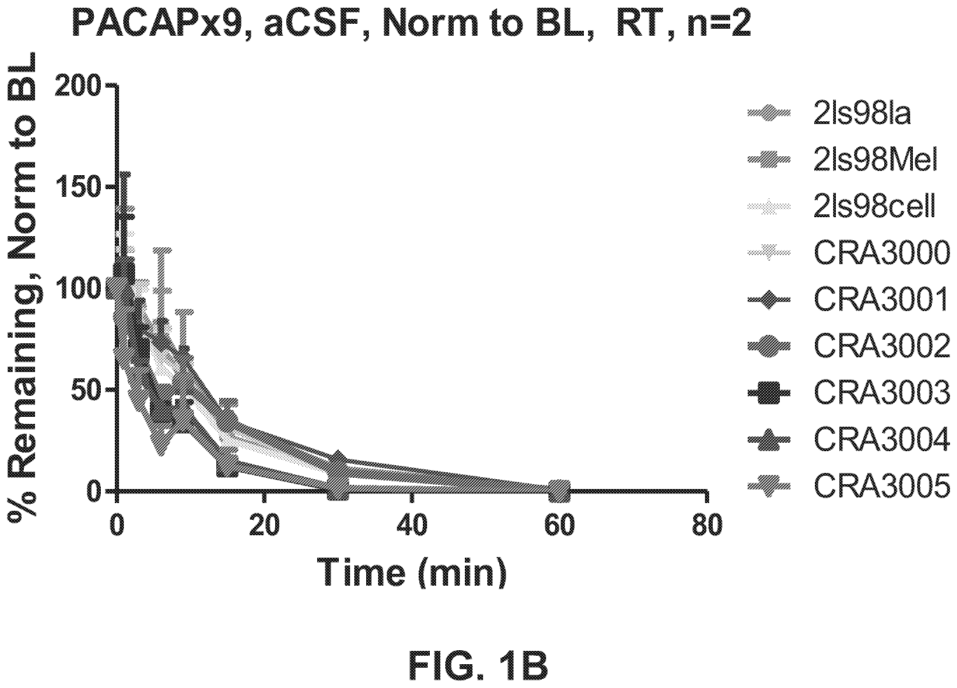

TABLE 1
Sequences in FIGS. 1A-1B

| Compound | Sequence |
|---|---|
| ID | 10 20 30 |
| PACAP27 | HSDGIFTDSY SRYRKQMAVK KYLAAVL |
| 2ls98LAC | HSDGIFTDSY SRYRKQLAVK KYLAAVL-Ser |
| | (Lactose) |
| 2ls98MEL | HSDGIFTDSY SRYRKQLAVK KYLAAVL-Ser |
| | (Melib10se) |
| 2ls98CEL | HSDGIFTDSY SRYRKQLAVK KYLAAVL-Ser |
| | (Cellob10se) |
| CRA3000 | HSDGIFTDSY SRYRKQÑAVK KYLAAVLŚ |
| CRA3001 | HSDGIFTDSY SRYRKQÑAVK KYLAAVŚ |
| CRA3002 | HSDGIFTDSY SRYRKQÑAVK KYLAAŚL |
| CRA3003 | HSDGIFTDSY SRYRKQÑAVK KYLAAVL\check{S} |
| CRA3004 | HSDGIFTDSY SRYRKQÑAVK KYLAAV\check{S} |
| CRA3005 | HSDGIFTDSY SRYRKQÑAVK KYLAA\check{S}L |
| CRAXXX | N-Acylat10n can be done with the active |
| | glycopeptides. Based on the binding and |
| | transport results from the first 6 |
| | glycopeptides, modificat10ns can also |
| | be made by the substitut10n of p-F- |
| | Phe(6), A(7), homo-Phe(6), Hyp(2) in |
| | addit10n to the original modificat10ns. |

s = D-Serine
Ñ = Norvaline
Ś = L-Serine-β-D-Glucoside
\check{S} = L-Serine-β-D-Lactoside

### DESCRIPTION OF PREFERRED EMBODIMENTS

As used herein, the natural amino acids refer to the twenty amino acids that are found in nature, i.e. occur naturally. The natural amino acids are as follows: alanine, arginine, glycine, asparagine, aspartic acid, cysteine, glutamine, glutamic acid, serine, threonine, histidine, lysine, methionine, proline, valine, isoleucine, leucine, tyrosine, tryptophan, and phenylalanine. This application adheres to the IUPAC rules of standard abbreviations for amino acids.

As used herein, the term “unnatural amino acids” refers to amino acids that are not naturally encoded or found in the genetic code of any organisms. Typically, the unnatural amino acids are different from the twenty naturally occurring amino acids in their side chain functionality. A non-limiting example of an unnatural amino acid is Norleucine (Nle).

Each amino acid may be either natural or unnatural of the “D” or “L” configuration which corresponds to the stereochemical designation “S” and “R,” respectively. As known to one of ordinary skill in the art, only L-amino acids are manufactured in cells and incorporated into proteins. The letter “D” preceding any abbreviation for an amino acid denotes the D-form of the amino acid, and a lack thereof refers to the L-form, unless specifically stated otherwise.

As defined herein, the term “agonist” refers to compound that enhances a response. The agonist binds to the same site as the endogenous compound and produces the same type of signal, usually of equal or greater magnitude than the endogenous agent. As defined herein, the term “antagonist” refers to compound that binds to the same site as the endogenous compound and diminishes or blocks the signal generated by the endogenous agent.

As used herein, the term “glycoside” is defined a molecule formed by a carbohydrate or a saccharide bound to another reactive functional group via a glycosidic bond, which is a covalent bond formed between the hemiacetal group of the carbohydrate and the reactive functional group, such as the hydroxyl group, of another compound. Glycosylation processes and glycans are well known to one of ordinary skill in the art. In some embodiments, the glycan is branched. In some embodiments, the glycan is unbranched. In some embodiments, the glycan is an N-linked glycan, or an O-linked glycan, or a C-linked glycan, or an S-linked glycan. Examples of glycans include, but are not limited to, glucose, linear or branched trisaccharides of glucose, lactose, maltose, cellobiose, melibiose, melibiose, glucosamine, N-acetylglucosamine, galactose, galactosamine, N-acetylgalactosamine, mannose, mannosamine, N-acetylmannos-amine, xylose, fucose, rhamnose, N-acetylneuraminic acid, N-glycolylneuraminic acid, 2-keto-3-deoxynononic acid, iduronic acid, and glucuronic acid. The present invention is not limited to the aforementioned glycans. For example, the glycan may be selected from all mono-, di-, tri- and poly-saccharides.

According to one embodiment, the present invention features a glycopeptide analog of PACAP₁₋₂₇ (SEQ ID NO: 1) or VIP₁₋₂₃ (SEQ ID NO: 2). In some embodiments, the glycopeptide analog may comprise a sequence according to any one of the following:

(SEQ ID NO: 3)
HSDGIFTDSY₁₀SRYRKQX¹AVK₂₀KYLAAVX²;
or
(SEQ ID 4)
HSDAVFTDNY₁₀TRLRKQX¹AVK₂₀KYLNSILN;

In some embodiments, X¹ may be M or norleucine or norvaline. Without wishing to be bound by a theory, when X¹ is norleucine, the glycopeptide analog can have an increased stability as compared to the glycopeptide analog where X¹ is M. In other embodiments, X² may be L or S. In some embodiments, the S₂ of SEQ ID NO: 3 or SEQ ID NO: 4 may be in a D or L configuration.

In preferred embodiments, at least one of the S residues in the sequence may be glycosylated with a glycan. For example, S₉ of SEQ ID NO: 3 may be glycosylated. As another example, S₁₁ of SEQ ID NO: 3 may be glycosylated. In one embodiment, X² may be S, and this S₂₇ of SEQ ID NO: 3 may be glycosylated. In another embodiment, S₂₅ of SEQ ID NO: 4 may be glycosylated. Without wishing to be bound by a theory or mechanism, the glycopeptide analog have an increased ability to cross a blood brain barrier (BBB) as compared to a peptide lacking glycosylation. Further still, the glycopeptide analog may be amphipathic.

In some embodiments, the glycan may be a saccharide, such as a mono-, di-, tri- or polysaccharide. In other embodiments, the glycan may be a glucose, a maltose, a melibiose, a lactose or a cellobiose. In still other embodiments, the glycan may be an O-linked glycan. For example, the glycan may be O-linked to the serine by bonding to the hydroxyl group in the side chain of serine.

In one embodiment, the glycopeptide analog may be a PAC₁ agonist. In another embodiment, the glycopeptide analog may be a VPAC₁ agonist. In a further embodiment, the glycopeptide analog may be a VPAC₂ antagonist.

TABLE 2 provides non-limiting examples of the sequences of the glycopeptide analogs of the present invention. In some embodiments, the S₂ of any of the sequences may be in a D or L configuration.

TABLE 2

| | SEQUENCE (S* refers to glycosylated |
|---|---|
| SEQ ID NO | S) |
| SEQ ID NO: 5 | HSDGIFTDS*Y₁₀SRYRKQMAVK₂₀KYLAAVL |
| SEQ ID NO: 6 | HSDGIFTDSY₁₀S*RYRKQMAVK₂₀KYLAAVL |
| SEQ ID NO: 7 | HSDGIFTDSY₁₀SRYRKQ-Nle-AVK₂₀KYLAAVL |
| SEQ ID NO: 8 | HSDGIFTDS*Y₁₀SRYRKQ-Nle-AVK₂₀KYLAAVL |
| SEQ ID NO: 9 | HSDGIFTDSY₁₀S*RYRKQ-Nle-AVK₂₀KYLAAVL |
| SEQ ID NO: 10 | HSDGIFTDSY₁₀SRYRKQ-Nle-AVK₂₀KYLAAVS |
| SEQ ID NO: 11 | HSDGIFTDS*Y₁₀SRYRKQ-Nle-AVK₂₀KYLAAVS |
| SEQ ID NO: 12 | HSDGIFTDSY₁₀S*RYRKQ-Nle-AVK₂₀KYLAAVS |
| SEQ ID NO: 13 | HSDGIFTDSY₁₀SRYRKQ-Nle-AVK₂₀KYLAAVS* |
| SEQ ID NO: 14 | HSDAVFTDNY₁₀TRLRKQMAVK₂₀KYLNS*ILN |
| SEQ ID NO: 15 | HSDAVFTDNY₁₀TRLRKQ-Nle-AVK₂₀KYLNSILN |
| SEQ ID NO: 16 | HSDAVFTDNY₁₀TRLRKQ-Nle-AVK₂₀KYLNS*ILN |

It may be appreciated that the glycopeptide analogs of the present invention can be utilized in pharmaceutical formulations and methods of treatment. Thus, in some aspects, the present inventions provides for pharmaceutical compositions that comprise any one of the glycopeptide analogs described herein, and methods of use thereof.

In one embodiment, the pharmaceutical composition may be effective for treating or preventing symptoms associated with Parkinson's disease. In another embodiment, the pharmaceutical composition may be effective for treating or preventing symptoms associated with degeneration of dopaminergic neurons of the substantia nigra pars compacta. In preferred embodiments, the pharmaceutical composition may a therapeutically effective amount of the glycopeptide analog of the invention. The composition may further comprise a pharmaceutically acceptable carrier. In some embodiments, the glycopeptide analog may be present in an amount ranging from 0.001 to 1.0 wt % of the composition. In exemplary embodiments, the composition may be in a form of a tablet, a nasal spray, or an intravenous solution.

According to some aspects, the present invention may feature a method of treating or preventing a symptom associated with Parkinson's diseing to the subject a therapeutically effective amount of a composition comprising ase in a subject. According to other aspects, the present invention may feature a method of treating or preventing a symptom associated with degeneration of dopaminergic neurons of the substantia nigra pars compacta. Said methods may comprise administerany of the glycopeptide analogs described herein. Without wishing to be bound by a theory or mechanism, the glycopeptide analog may be configured to cross through the BBB.

In some embodiments, the composition being administered may further comprise a pharmaceutically acceptable carrier. In other embodiments, the subject may be a mammal, such as a human. In one embodiment, the glycopeptide analog may be administered in a dosage of about 0.001 mg/kg to 100 mg/kg of body weight, or any range in between. In another embodiment, the composition may be administered daily, weekly, or monthly. In further embodiments, the composition is administered intranasally, intravenously, transdermally, or orally.

As used herein, the terms “treat”, “treating”, or “treatment” refer to both therapeutic treatment and prophylactic or preventative measures, with the objective of preventing, reducing, slowing down (lessen), inhibiting, or eliminating an undesired physiological change, symptom, disease, or disorder, such Parkinson's disease. For purposes of this invention, beneficial or desired clinical results include, but are not limited to, alleviation (e.g. reduction, lessening, or inhibition) of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition (e.g., Parkinson's disease) prior to administration of the peptide analog of the invention. Subjects at risk for Parkinson's disease can be identified by, for example, any or a combination of appropriate diagnostic or prognostic assays known in the art.

A “therapeutically effective amount” refers to an amount that is sufficient to achieve the desired therapeutic result or to have an ameliorating effect on undesired symptoms, but is generally insufficient to cause adverse side effects. The specific therapeutically effective dose level for any particular patient will depend upon a variety of factors including the disease being treated and the severity of the disease; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

The terms “administering” and “administration” refer to methods of providing a pharmaceutical composition to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, topically or the like. Administration of the composition can occur, daily, weekly, monthly, or any period in between. In some embodiments, the composition may be administered periodically for a set period of time, e.g. once per week for between about 1 to 10 weeks. The compound may also be administered chronically throughout a subject's lifetime. One skilled in the art would recognize how to monitor the effectiveness of the treatment and how to adjust the treatment accordingly. A skilled artisan will appreciate that certain factors may influence the dosage and timing required to effectively treat a subject, including but not limited to the severity of the disease or disorder, previous treatments, the general health and/or age of the subject, and other diseases present. If a subject does not respond to the initial dosage and administration of the composition, a person of skill can administer the medication daily for several days until a desired response occurs. A person of skill can monitor a subject's clinical response to the administration of the composition, and administer additional dosages or increase the dosages as needed.

As described above, the compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Depending on the intended mode of administration, the pharmaceutical compositions may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. The compositions will include, as noted above, an effective amount of the selected glycopeptide analog drug in combination with a pharmaceutically acceptable carrier and, in addition, may include other medicinal agents, pharmaceutical agents, carriers, adjuvants, diluents, etc.

In some embodiments, solid compositions may comprise conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art.

In some embodiments, for oral administration, fine powders or granules may contain diluting, dispersing, and/or surface active agents, and may be presented in water or in a syrup, in capsules or sachets in the dry state, or in a non-aqueous solution or suspension where suspending agents may be included, in tablets where binders and lubricants may be included, or in a suspension in water or a syrup. Where desirable or necessary, flavoring, preserving, suspending, thickening, or emulsifying agents may be included. Tablets and granules may be coated.

Parenteral administration is generally characterized by injection. Common parenteral routes are intramuscular (IM), subcutaneous (SC) and intravenous (IV). Injectables can be prepared in conventional forms, either as liquid solutions or suspensions, solid forms suitable for solution or suspension in liquid prior to injection, or as emulsions. In some embodiments, parental administration may involve use of a slow release or sustained release system, such that a constant level of dosage is maintained.

In other embodiments, the compositions may be administered topically. For topical administration, liquids, suspension, ointments, lotions, creams, gels, drops, suppositories, sprays, powders or the like may be used as long as the active compound can be delivered through the surface of the skin. Conventional pharmaceutical carriers, aqueous, powder or oily bases, thickeners and the like may be necessary or desirable.

### EXAMPLES

The following are non-limiting examples of practicing the present invention. It is to be understood that these examples are not intended to limit the invention in any way, and that equivalents or substitutes are within the scope of the invention.

### Example 1: Synthesis of Peptides and Glycopeptides

The glycopeptide analogs were synthesized using methods for the incorporation of the glycosides. All of the peptides and glycopeptides used in this study were highly purified (>97% based on HPLC analysis).

Resin preparation. One gram (1.00 g) of Rink amide-MBHA resin (0.5 mmol, substitution=0.5 mmol/g) was placed into a 12 mL fritted syringe. The resin was washed with 6 mL DMF and placed on a slow tumbler for 2 minutes. The DMF was expelled, and the washing was repeated with an additional 6 mL DMF.

Fmoc deprotection. A mixture of organic bases 2% DBU-2% piperidine in DMF (6 mL) was added to the resin and the syringe was tumbled for 5 min. The organic base mixture was expelled and the cleavage treatment was repeated for an additional 10 min with fresh base. The free NH₂ resin was suspended in fresh DMF (6 mL) and the syringe was tumbled for 2 minutes. The DMF was expelled and the washing was repeated 4× with DMF. The resin was washed a final time with NMP the same manner.

Fmoc amino acid glycoside peracetate coupling. 0.65 mmol, (1.3 eq.) [N-(9-fluorenylmetoxycarbonyl)-L-serine-3-yl] peracetyl-β-O-glycopyranoside and 0.65 mmol, (1.3 eq.) HOBT were placed into a 20 mL vial and dissolved in 6 mL NMP. Into the solution 0.65 μmol, (1.3 eq.) DIC was added, and the mixture was shaken for 1 minute, then added to the resin. The syringe with the resin and activated amino acid was tumbled for 5 minutes, then it was placed to the center of the rotating plate of 1250 Watt commercial microwave oven. The oven was set to Power Level 1 (intermittent heating) for 10 minutes. During this time, every 30 seconds the syringe was shaken manually for 10 seconds and returned to the microwave for a total of 10 minutes. The solvent was expelled from the syringe and the resin was washed once initially with NMP and 5× with DMF in the same manner as described above.

The Fmoc protecting group was removed as described above, and the DMF washing protocol was repeated, ending with the NMP wash.

Fmoc amino acid coupling. Subsequent amino acid couplings were accomplished on the Prelude® synthesizer with 3 eq HBTU, 12 eq of NMM and 3 eq of the desired amino acid in 14 mL of DMF in double coupling mode. After coupling, the resin was washed 6× with 10 mL DMF for 2 minutes. The Fmoc removal was accomplished as above, but the washing was 6× with 10 mL DMF for 2 minutes. After the last Fmoc cleavage, the acetyl groups were removed.

Acetate cleavage. The wet (DMF) resin was treated with 10 mL of 50% H₂NNH₂.H₂O in DMF for 2×30 minutes, and 1×60 minutes, followed by 6× washes with 10 mL DMF, 2× with 10 mL CH₃OH, 4× with 10 mL DMF and 6× with 10 mL CH₂Cl₂, followed by drying in vacuo.

Peptide cleavage. Each batch of dried peptide resin was cleaved in a fritted syringe in which it was assembled using 10 mL of a “cleavage cocktail” (9.0 mL of TFA, 1.0 mL of CH₂Cl₂, 0.25 mL of Et₃SiH, 0.25 mL of H₂O, and 0.05 mL anisole) for 1 hour at RT. After cleavage the solution was expelled and the resin was washed 2× with 4 mL of the cleavage cocktail, and the combined solutions were concentrated to −5 mL (oil) by a stream of dry N₂. Cold Et₂O (35 mL) was poured over the peptide solutions to precipitate each product. The crude glycopeptides were centrifuged, dried in vacuo, then re-dissolved in H₂O, freeze-dried, analyzed by analytical HPLC and separated by preparative HPLC. The appropriate fractions were freeze-dried to provide amorphous white powders. Yields of the purified peptides/glycopeptides ranged from 14-39%.

Analytical HPLC conditions. Varian ProStar® HPLC system. Mobil phase: 1 mL/min flow rate, gradient 100% solvent A to 100% solvent B over 20 minutes. Solvent A is 5% CH₃CN in H₂O with 0.1% TFA, B solvent is 80% CH₃CN in H₂O with 0.1% TFA. Column: Dikma Technology, Inspire™, C₁₈, 5 μm, 250×4.6 mm. Detection at 280 nm.

Preparative HPLC conditions. Gilson Preparative HPLC system. Mobil phase: 25 mL/min flow rate, gradient 100% A solvent to 100% B solvent over 60 minutes. Solvents A and B are as in the analytical HPLC system. Column: Phenomenex, Luna C₁₈, 10 μm, 250×50 mm. Detection at 280 nm.

### Example 2: Shotgun Microdialysis

A persistent challenge in animal research is the control for all the variables in complex biological systems, even genetically identical animals do not react identically. Different animals often have different responses to the same treatment, and it is difficult to control for variations in the injection site between animals, often leading to large numbers of animals being used to produce a statistically valid result. We have developed an alternative method which controls for intra-animal and injection site variation, and reduces the number of animals required. We have coined this technique “shotgun microdialysis,” in which we inject a single animal with multiple compounds of interest and monitor the CSF concentrations using microdialysis. This allows us to directly compare the compounds within a single animal, with a single injection site, and account for the variability between animals.

In microdialysis, a probe with a semi-permeable membrane is surgically implanted into the target region (the striatum in this case), and the perfusate flows through the probe, allowing molecular species of the appropriate molecular weights (our drug candidates) to diffuse across the membrane as a function of concentration gradient. Microdialysis allows for sampling from a region without altering the volume of that region, which is of particular relevance when studying the CSF

Animals for serum stability in vivo, and CSF microdialysis studies. Male Sprague-Dawley rats (275-325 grams) may be used for in vivo experiments. Animals can be purchased from Harlan Laboratories (Indianapolis, Ind.) and housed in a temperature and humidity controlled room with 12 h reversed light/dark cycles with food and water available ad libitum. All animals are treated as approved by the Institutional Animal Care and Use Committee, University of Arizona and in accordance with the NIH Guidelines for the Care and Use of Laboratory Animals. Both the number of animals used and their suffering are minimized.

Compounds are spiked into rat serum at 37° C. and monitored over time. Aliquots are quenched with acetic acid and spiked with internal standard (dAdLE), then desalted with C¹⁸ ZipTips®. Samples are run in duplicate with direct injection using a 20-μL injection loop. Quantification was based on the area under the curve (AUC) for the identified MS2 fragments over the course of the injection.

Probe recovery studies are performed with a series of microdialysis probes to optimize for the best recovery of the compounds. Probes are submerged in a stirring solution of the glycopeptides analog compounds in aCSF and recovery was calculated by comparing the solution concentration to the concentration of the dialysate, accounting for dilution. As microdialysis is inherently a diffusion-limited technique, there is a delicate balance between probe recovery and temporal resolution. The slower the perfusate is flowed through the probe, the higher the recovery will be, but the sampling time is increased. A flow rate of 0.5 μL/minute may be used to optimize both percent recovery and temporal resolution. The pairing of a second line that introduces preservation solution can yield a temporal resolution of 10 minutes for the collection of 10 μL of solution. Increasing the flow rate would increase the temporal resolution but would also decrease the percent recovery.

To directly compare the in vivo lifetime and BBB penetration of the glycopeptides analogs, a method that involves dual blood draws and microdialysis may be used. The compounds can be injected intravenously at 10 mg/kg via a single tail vein injection. Blood draws can be taken at t=−10, 1, 5, 10, 20, 30, 40, 50, 60, 70, 80, and 90 minutes (where t=0 corresponds to injection). Microdialysis fractions are time-locked with blood draws at ten-minute increments such that blood draws correlate with the median time of the microdialysis fraction. Blood draws are centrifuged for 2 minutes in a tabletop mini-centrifuge to separate the serum and the red blood cells. Serum is pulled off and diluted 100× into a solution of 50:50 aCSF:“preservation solution” for matrix matching with the dialysate and standards, and must be immediately frozen on dry ice. Dialysate samples are collected for 10 minutes at a flow rate of 0.5 μL/min, with the microdialysis tee coupling a line containing preservation solution to the dialysate line immediately behind the probe, leading to a solution volume of 10 μL after 10 minutes. The dialysate samples must be immediately frozen on dry ice. Post-experiment, samples should be stored in a −80° C. freezer until analysis.

All samples may be desalted using μ-C₁₈ Zip Tips® (EMD Millipore). ZipTip® cleanup is a common practice in proteomics analysis, as it is highly useful for desalting biological samples. However, C₁₈ ZTs may have a poor recovery for our targets of interest, due to their hydrophilicity. In order to improve the recovery but maintain the benefit of desalting, the pairing agent octyl sulfonate (8S) may be added. Octyl sulfonate may increase the recovery of the glycopeptides analogs during the ZipTip® desalting process. Octyl sulfonate can ionically pair with the compounds and its effects can last through ZT and column separations, but is removed during ionization so that the masses of the compounds are not altered.

The serum concentration and CSF concentration estimates can be calculated using a calibration curve, accounting for the dilution factors and the probe recoveries for each experiment. Standards may be matrix-matched to samples and undergo the same sample preparation steps. Probe recoveries for probes used in the in vivo experiments may be calculated by submerging the probe into a vial of standards post-experiment. Note that the probe recoveries are highly variable between probes.

### Example 3: Mass Spectrometric Identification

In vitro studies can be performed using an Applied Biosystems QStar Elite mass spectrometer, using quantitation of MS² fragments. For the in vivo studies, a Proxeon nano-LC coupled to a Thermo LTQ-Orbitrap instrument may be used. MS³ fragments may be quantified for increased specificity. The high number of small peptides present in biological solutions such as blood, paired with the minimal clean-up applied with to the method, means that the matrix is highly complex and high specificity is required to differentiate our small peptides from the hundreds that are present. Both retention time and MS³ fragment identification are necessary to assure that the target molecules are being quantified.

### Example 3: Assay Binding of Peptides to PAC and VPAC Receptors

| Drug | Sequence |
|---|---|
| PACAP1-27 | HSDGIFTDSYSRYRKQMAVKKYLAAVL |
| 2ls98Cell | HSDGIFTDSYSRYRKQLAVKKYLAAV_{d8}L-Ser(Cell) |
| 2ls98Lact | HSDGIFTDSYSRYRKQLAV_{d8}KKYLAAV_{d8}L-Ser(Lact) |
| 2ls98Mel | HSDGIFTDSYSRYRKQLAVKKYLAAVL-Ser(Mel) |
| CRA3000 | HsDGIFTDSYSRYRKQÑAVKKYLAAVL-Ser(Glc) |
| CRA3001 | HsDGIFTDSYSRYRKQÑAVKKYLAAV-Ser(Glc) |
| CRA3002 | HsDGIFTDSYSRYRKQÑAVKKYLAAL-Ser(Glc)-L |
| CRA3003 | HsDGIFADSYSRYRKQÑAVKKYLAAVL-Ser(Glc) |
| CRA3004 | HsDGIFADSYSRYRKQÑAVKKYLAAV-Ser(Glc) |
| CRA3005 | HsDGIFADSYSRYRKQÑAVKKYLAAV-Ser(Glc)-L |

| | PAC1 | | VPAC1 | | VPAC2 | |
|---|---|---|---|---|---|---|
| | EC₅₀ | E_{Max} | EC₅₀ | E_{Max} | EC₅₀ | E_{Max} |
| Drug | (nM) | (%) | (nM) | (%) | (nM) | (%) |
| PACAP1-27 | 0.4, 0.13, 0.34 | 100 | 14.8 ± 1.6 | 100 | 0.35 ± 0.16 | 100 |
| 2ls98Cell | 0.84 | 92 | 0.52 | 93 | 55.6 | 91 |
| 2ls98Lact | 0.72 | 93 | 0.45 | 101 | 193 | 100 |
| 2ls98Mel | 0.57 | 99 | 0.55 | 102 | 9.4 | 86 |
| CRA3000 | 25.5 | 85 | 1.3 | 90 | 241 | 104 |
| CRA3001 | 54.5 | 86 | 4.8 | 90 | 654 | 107 |
| CRA3002 | >250 | −79 | 5.9 | 93 | >2500 | −95 |
| CRA3003 | >250 | −71 | 78.8 | 93 | >2500 | −42 |
| CRA3004 | NC | NC | 1366 | 85 | NC | NC |
| CRA3005 | NC | NC | 1623 | 78 | NC | NC |

### Example 4: Preparation of a Pharmaceutical Composition

10 g of a glycopeptide analog is mixed with 1900 g of an aqueous solution comprising cellulose, polyvinylpyrrolidone, and sucrose. The glycopeptide analog is according to any one of the sequences described herein. This liquid mixture is passed through granulating sieves and desiccated for at least 24 hours at room temperature to produce a dry mixture. The dry mixture should then be compressed into tablets of desired weight and physical specifications by methods known to those skilled in the art. For instance, the dry mixture is formed into tablets, each weighing 100 mg with an available dose of 0.05 mg of the glycopeptide analog.

### Example 5: Treatment Study of Parkinson's Disease with the Pharmaceutical Composition Described in Example 6 as Follows

A male Parkinsonian monkey exhibits symptoms of pronounced tremors and bradykinesia. The following treatment is administered to the Parkinsonian monkey: In the first period of treatment, two tablets per day for two weeks are orally administered, followed by a one-week off period, which completes one cycle. After the off period, the cycle is repeated for a total treatment time of 3 months. The Parkinsonian monkey exhibits improvement of motor symptoms and balance and the dyskinesias is significantly reduced. Three independent repetitions of the study are performed.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

1. A glycopeptide analog of pituitary adenylate cyclase activating peptide (SEQ ID NO: 1) or vasoactive intestinal peptide (SEQ ID NO: 2), comprising a sequence according to any one of the following:
(SEQ ID NO: 3)
HSDGIFTDSY₁₀SRYRKQX¹AVK₂₀KYLAAVX²;
or
(SEQ ID NO: 4)
HSDAVFTDNY₁₀TRLRKQX¹AVK₂₀KYLNSILN;
wherein X¹ is M, norleucine, or norvaline, wherein X² is L or S, and wherein at least one of the S residues in the sequence is glycosylated with a glycan.

2. The glycopeptide analog of claim 1, wherein S₉ of SEQ ID NO: 3 is glycosylated.

3. The glycopeptide analog of claim 1, wherein S₁₁ of SEQ ID NO: 3 is glycosylated.

4. The glycopeptide analog of claim 1, wherein X² is S, wherein this S₂₇ of SEQ ID NO: 3 is glycosylated.

5. The glycopeptide analog of claim 1, wherein S₂₅ of SEQ ID NO: 4 is glycosylated.

6. The glycopeptide analog of any of claim 1, wherein S₂ of SEQ ID NO: 3 or SEQ ID NO: 4 is in a D or L configuration.

7. The glycopeptide analog of claim 1, wherein the glycan is a saccharide.

8. The glycopeptide analog of claim 7, wherein the saccharide is a mono-, di-, tri- or poly-saccharide.

9. The glycopeptide analog of claim 1, wherein the glycan is a glucose, a maltose, a melibiose, a lactose or a cellobiose.

10. The glycopeptide analog of claim 1, wherein the glycan is an O-linked glycan.

11. The glycopeptide analog of claim 1, wherein the glycopeptide analog is amphipathic.

12. The glycopeptide analog of claim 1, wherein the glycosylated peptide has an increased ability to cross a blood brain barrier (BBB) as compared to a peptide lacking glycosylation.

13. The glycopeptide analog of claim 1, wherein when X¹ is norleucine, the glycopeptide analog has an increased stability as compared to the glycopeptide analog where X¹ is M.

14. The glycopeptide analog of claim 1, wherein the glycopeptide analog is a PAC₁ agonist.

15. The glycopeptide analog of claim 1, wherein the glycopeptide analog is a VPAC₁ agonist.

16. The glycopeptide analog of claim 1, wherein the glycopeptide analog is a VPAC₂ antagonist.

17. A glycopeptide analog selected from a group consisting of:

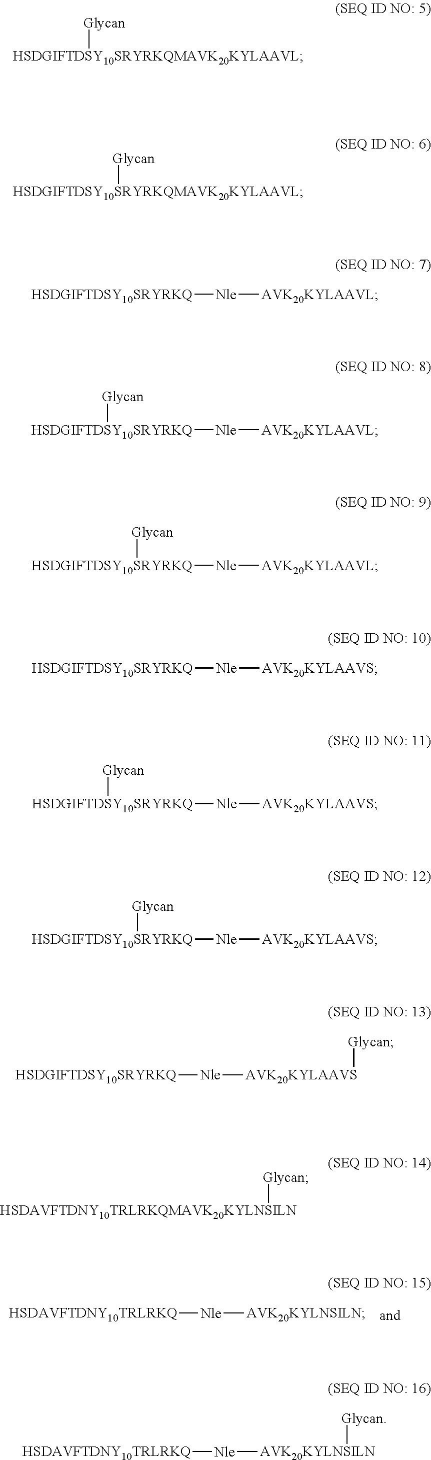

18. The glycopeptide analog of claim 17, wherein the glycan is a saccharide.

19. The glycopeptide analog of claim 18, wherein the saccharide is a mono-, di-, tri- or poly-saccharide.

20. The glycopeptide analog of claim 17, wherein the glycan is a glucose, a maltose, a melibiose, a lactose or a cellobiose.

21.-34. (canceled)
